(19) United States
(12) Patent Application Publication
(10) Pub. No.: US 2008/0118541 A1
(43) Pub. Date: May 22, 2008

(54) USE OF A TERPOLYMER OF TETRAFLUOROETHYLENE, HEXAFLUOROPROPYLENE, AND VINYLIDENE FLUORIDE IN DRUG ELUTING COATINGS ON MEDICAL DEVICES

(75) Inventor: Stephen Pacetti, San Jose, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 11/562,338

(22) Filed: Nov. 21, 2006

(51) Int. Cl. A61F 2/02; A61L 27/00; C08F 16/24

(52) U.S. Cl. 424/423; 427/2.24; 526/206; 526/247; 623/23.36

(57) ABSTRACT

Medical devices are coated with terpolymers of tetrafluoroethylene, hexafluoropropylene, and vinylidene fluoride (THV). The mole fraction of tetrafluoroethylene can be in a range from about 0.005 to about 0.85, the mole fraction of hexafluoropropylene monomer can be in a range from about 0.005 to about 0.85, and the mole fraction of vinylidene fluoride can be in a range from about 0.005 to about 0.99. One example method of applying the terpolymers to a medical device includes dissolving the terpolymers in a solvent and applying the solution to the medical device and then removing the solvent. The THV coating on the implantable medical devices are advantageously biocompatible.

### RELATED APPLICATIONS

This application is related to co-pending U.S. Provisional Patent Application No. ______, entitled “Copolymers Having Zwitterionic Moieties And Dihydroxyphenyl Moieties And Medical Devices Coated With The Copolymers” (Attorney Docket No. 16497.62), co-pending U.S. Provisional Patent Application No. ______, entitled “Methods of Manufacturing Copolymers with Zwitterionic Moieties and Dihydroxyphenyl Moieties and Use of Same” (Attorney Docket No. 16497.63), co-pending U.S. Provisional Patent Application No. ______, entitled “Zwitterionic Copolymers, Method of Making and Use on Medical Devices” (Attorney Docket No. 16497.64), co-pending U.S. Provisional Patent Application No. entitled “Zwitterionic Terpolymers, Method of Making and Use on Medical Devices” (Attorney Docket No. 16497.65), co-pending U.S. Provisional Patent Application No. entitled “Amino Acid Mimetic Copolymers and Medical Devices Coated with the Copolymers” (Attorney Docket No. 16497.70), co-pending U.S. Provisional Patent Application No. ______, entitled “Methods for Manufacturing Amino Acid Mimetic Copolymers and Use of Same” (Attorney Docket No. 16497.71), co-pending U.S. Provisional Patent Application No. ______, entitled “Copolymers Having 1-Methyl-2-Methoxyethyl Moieties” (Attorney Docket No. 16497.72), and co-pending U.S. Provisional Patent Application No. ______, entitled “Methods for Manufacturing Copolymers Having 1-methyl-2-Methoxyethyl Moieties and Use of Same” (Attorney Docket No. 16497.73), each of which was filed Nov. 21, 2006, and each of which is hereby incorporated by reference in their entirety.

### BACKGROUND OF THE INVENTION

1. The Field of the Invention

Embodiments of the invention relate to polymer coated implantable medical devices. More particularly, embodiments of the invention relate to implantable medical devices coated with terpolymers of tetrafluroethylene(TFE), hexafluropropylene (HFP), and vinylidene fluoride (VDF).

2. The Related Technology

Implantable intravascular stents are commonly used in many medical procedures to treat disorders of the circulatory system. Although these devices work well mechanically, chronic issues of restenosis and, to a lesser extent, thrombosis remain. These biologically derived issues are currently being addressed using pharmacological therapies, including the use of drug eluting polymer coatings on stents. Polymeric coatings used on implantable medical devices for drug delivery typically serve two purposes. First, the polymer holds the drug on the device such that it is presented to the lesion. Secondly, the polymer controls the release rate of the drug from the coating to maintain an efficacious tissue concentration for the duration of time required to yield the clinically desired result.

In addition to these primary roles for drug delivery, the materials used in coating implantable vascular stents should satisfy additional criteria including: adhesion to the implant (e.g. adhesion to stent struts) to prevent delamination; adequate elongation to accommodate implant deformation without buckling or cracking; sufficient hardness to withstand crimping operations without excessive damage; sterilizability; biocompatibility including hemocompatibility and chronic vascular tissue compatibility; in the case of durable or permanent coatings, the polymer needs to be sufficiently biostable to avoid biocompatibility concerns; processability (e.g. production of stent coatings that are microns thick); reproducible and feasible polymer synthesis; and an adequately defined regulatory path.

One class of polymers extensively used in implantable medical devices is fluoropolymers. One common example is poly(tetrafluoroethylene) (Teflon®) which is used in vascular grafts and soft tissue implants. Fluoropolymers possess many properties that render them useful for coatings on implantable devices. For example, fluoropolymers have excellent biostability, good blood compatibility, and low water absorption, which enables low drug permeability for good drug release control.

One problem with existing fluoropolymers used to coat implantable medical devices is their high degree of crystallinity. The high crystallinity of the polymers makes the polymers difficult to process and apply to a medical device. In addition, high crystallinity causes poor elongation, which can lead to cracking of the polymer coating during use. Lastly, a high degree of crystallinity can cause the diffusivity of the drug in the polymer to be too low.

### SUMMARY OF THE INVENTION

The implantable medical devices of embodiments of the invention are coated with poly(tetrafluoroethylene-co-hexafluoropropylene-co-vinylidene fluoride) (hereinafter “THV”). In an embodiment, the THV polymer has the following chemical formula.

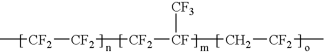

### In the foregoing formula, n is in a range from 0.005 to 0.85, m is in a range from 0.005 to 0.85, and o is in a range from 0.005 to a 0.99.

Coating the implantable medical devices with THV is advantageous for several reasons. One advantage of THV is its elasticity. THV has superior elongation properties compared to many fluoropolymers and other hydrophobic polymers used on medical devices. Good polymer elongation is beneficial for avoiding polymer cracking during application of the polymer and use of the device. Another advantage is that THV includes some tetrafluoroethylene monomer. Poly(tetrafluoroethylene) is one of the most chemically resistant, stable, and lubricious polymers available. To the extent that the TFE monomer is present at the surface, a coating of THV can be more lubricious and inert compared to other coatings.

Another advantage of THV is its processability. THV is soluble in several organic solvents. Consequently, THV can be applied to the implantable device using solvent based techniques including, but not limited to, spraying, dip coating, roll coating, spin coating, direct application by brush or needle, inkjet printing, or the like. Solvent-based application techniques are useful for applying a thin, even coating, which can be advantageous for controlled drug delivery. In an alternative embodiment, the THV polymers can be applied to a medical device using non-solvent techniques including powder coating. One skilled in the art will appreciate the many different techniques in powder coating.

These and other advantages and features of the invention will become more fully apparent from the following description and appended claims, or may be learned by the practice of the invention as set forth hereinafter.

### BRIEF DESCRIPTION OF THE DRAWINGS

To further clarify the above and other advantages and features of the invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings, in which:

FIG. 1A illustrates a stent coated with a THV terpolymer according to one embodiment of the invention; and

FIG. 1B is a cross-section of a strut of the stent of FIG. 1A.

### DETAILED DESCRIPTION OF EMBODIMENTS OF THE INVENTION

### I. TERPOLYMERS

Embodiments of the invention relate to implantable medical devices coated with terpolymers of tetrafluoroethylene, hexafluoropropylene, and vinylidene fluoride (THV). The implantable medical devices coated with THV provide superior performance and improved manufacturability compared to existing implantable medical devices coated with fluoropolymers. In an embodiment, the THV polymer has the following chemical formula.

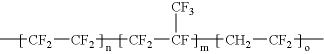

In the foregoing formula, n is in a range from about 0.005 to about 0.85, m is in a range from about 0.005 to about 0.85, and o is in a range from about 0.005 to about 0.99. Unless otherwise stated, the monomers shown in the chemical formula above and other chemical formulas herein can be in any order within the copolymer molecule and the monomer linkages shown in the chemical formulas only represent that the monomers are part of the same copolymer molecule. Furthermore, unless otherwise stated, the polymeric molecules can include monomers other than those shown in the chemical formulas.

The THV coating on the implantable medical devices of the invention are advantageously biocompatible. The THV polymer includes only fluorinated monomers that have been shown to be biocompatible when used as a homopolymer and/or copolymer on medical devices. For example, poly(tetrafluoroethylene) has been used on vascular grafts and poly(vinylindene fluoride) and poly(vinylidene flouride-co-hexafluoropropylene) have been used in implantable sutures.

Another feature of the THV polymer is that it can be soluble in an organic solvent. This feature is in contrast to most polymers that include tetrafluoroethylene monomers, which are typically insoluble in organic solvents. Solvent insoluble fluoropolymers include poly(tetrafluoroethylene-co-hexafluoropropylene), poly(tetrafluoroethylene-co-ethylene), and poly(tetrafluoroethylene-co-chlorotrifluoroethylene. These polymers are insoluble in organic solvents, in large part because of the crystallinity of the TFE monomer.

The THV used in embodiments of the invention can be solvent soluble because of the hexafluoropropylene and vinylidene fluoride monomers. As a homoploymer, poly(vinylidene fluoride) is solvent soluble due to the high dipolar moment of the CF₂ group. As a hompopolymer, or when polymerized under free radical conditions with other monomers, the hexafluoropropylene monomer leads to an amorphous polymer due to its atactic structure. Consequently, hexafluoropropylene and vinylidene fluoride monomers inhibit crystallization of the tetrafluoroethylene. To achieve solvent solubility, the THV monomer can include less than 75 mole % of tetrafluoroethylene monomer and in an alternative embodiment less than 50 mol %.

The physical properties of various commercially available THV polymers (available from Dyneon) are shown in Table 1.

TABLE 1
Physical Properties of THV¹

| | Melting | Tensile at | Elongation | Flexural | Hardness, |
|---|---|---|---|---|---|
| | Point | Break | at | Modulus | Shore |
| Polymer | (° C.) | (MPa) | Break | (MPa) | D |
| THV 220A | 120 | 20 | 600 | 80 | 44 |
| THV 415 | 155 | 28 | 500 | 180 | 53 |
| THV 500A | 165 | 28 | 500 | 210 | 54 |
| PBMA | none | 10 | 300 | NA | NA |
| (for | | | | | |
| comparison) | | | | | |

The physical properties of the Dyneon THV polymers in Table 1 illustrate various properties that make THV suitable for use on implantable medical devices, particularly for drug eluting stents. For comparison to the Dyneon THV polymers, Table 1 also includes poly(n-butyl methacrylate) (PMBA), which is currently being used on drug eluting stents such as Xience V™ and CYPHER™.

As shown in Table 1, the ultimate elongations for THV are high and show that the THV polymers can plastically deform without cracking. In one embodiment, the THV polymer has an elongation in a range from about 50% to about 800%, alternatively in a range from about 100% to about 700%. In yet another alternative embodiment, the elongation is in a range from about 300% to about 800% and alternatively in a range from about 400% to about 700%.

In contrast to PBMA, the THV polymers have a melting point. The existence of a melting point indicates that the THV polymers have some crystallinity, but not so much as to prevent solubility. A small amount of crystallinity is advantageous because it gives the polymer strength.

The THV polymers can be synthesized by a free radical process using either suspension or emulsion polymerization. Typical initiators are peroxide and azo compounds, organic soluble peroxides being used advantageously for suspension polymerization. The reaction is performed in an autoclave due to the gaseous nature of the monomers and water is the most common dispersed phase. The polymerization is a single-step reaction with minimal purification required as the gaseous monomers escape once the reactor is vented to the atmosphere. Examples of THV polymers suitable for use in embodiments of the invention are commercially available from Dyneon, LLC (Oakdale, Minn.).

The polymerization reaction can be controlled to produce the copolymers of the invention with a desired molecular weight. In one embodiment, the number average molecular weight of the copolymer is in the range from about 20K to about 800K, in another embodiment, the number average molecular weight is in a range from about 100K to about 600K.

### II. METHOD OF COATING IMPLANTABLE DEVICES AND METHODS OF USE

The implant devices of embodiments of the invention can be coated with THV using solvent and non-solvent based techniques. Examples of suitable techniques for applying the coating to the medical device include spraying, dip coating, roll coating, spin coating, powder coating, inkjet printing, and direct application by brush or needle. The copolymers can be applied directly to the surface of the implant device, or they can be applied over a primer or other coating material.

The THV polymers can be used alone as a coating or can be combined with other polymers or agents to form a polymer coating. The THV polymers can be used as a base coat, top coat, or other coating layer and can be used with or without a primer coating.

In one embodiment, the polymer coatings are applied to a medical device using a solvent-based technique. The polymer can be dissolved in the solvent to form a solution, which can be more easily applied to the medical device using one or more of the above mentioned techniques or another technique. Thereafter substantially all or a portion of the solvent can be removed to yield the polymer coating on a surface of the medical device.

Examples of suitable solvents that can be used with the copolymers of the invention include, but are not limited to, dimethylacetamide (DMAC), dimethylformamide (DMF), tetrahydrofuran (THF), dimethylsulfoxide (DMSO), cyclohexanone, xylene, toluene, acetone, i-propanol, methyl ethyl ketone, propylene glycol monomethyl ether, methyl t-butyl ketone, methyl isobutyl ketone, ethyl acetate, n-butyl acetate, n-butanol, ethanol, methanol, chloroform, trichloroethylene, 1,1,1-trichloreoethane, methylene chloride, cyclohexane, and dioxane. Solvent mixtures can be used as well. Representative examples of the mixtures include, but are not limited to, DMAC and acetone (50:50 w/w); tetrahydrofuran and DMAC (80:20, 50:50, or 20:80 w/w); and acetone and cyclohexanone (80:20, 50:50, or 20:80 w/w).

Examples of suitable implantable devices that can be coated with the copolymers of the invention include coronary stents, peripheral stents, catheters, arterio-venous grafts, by-pass grafts, pacemaker and defibrillator leads, anastomotic clips, arterial closure devices, patent foramen ovale closure devices, and drug delivery balloons. The copolymers are particularly suitable for permanently implanted medical devices.

The implantable device can be made of any suitable biocompatible materials, including biostable and bioabsorbable materials. Suitable biocompatible metallic materials include, but are not limited to, stainless steel, tantalum, titanium alloys (including nitinol), and cobalt alloys (including cobalt-chromium-nickel and cobalt-chromium-tungsten alloys). Suitable nonmetallic biocompatible materials include, but are not limited to, polyamides, fluoropolymers, polyolefins (i.e. polypropylene, polyethylene etc.), nonabsorbable polyesters (i.e. polyethylene terephthalate), and bioabsorbable aliphatic polyesters (i.e. homopolymers and copolymers of lactic acid, glycolic acid, lactide, glycolide, para-dioxanone, trimethylene carbonate, ε-caprolactone, and the like, and combinations of these).

The THV polymer is particularly useful as a coating for stents due to its biocompatibility, elongation, mechanical strength, and controlled drug release. The THV polymer coated stents can be self-expanding or balloon expandable. The stents can be composed of wire structures, flat perforated structures that are subsequently rolled to form tubular structures, or cylindrical structures that are woven, wrapped, drilled, etched or cut.

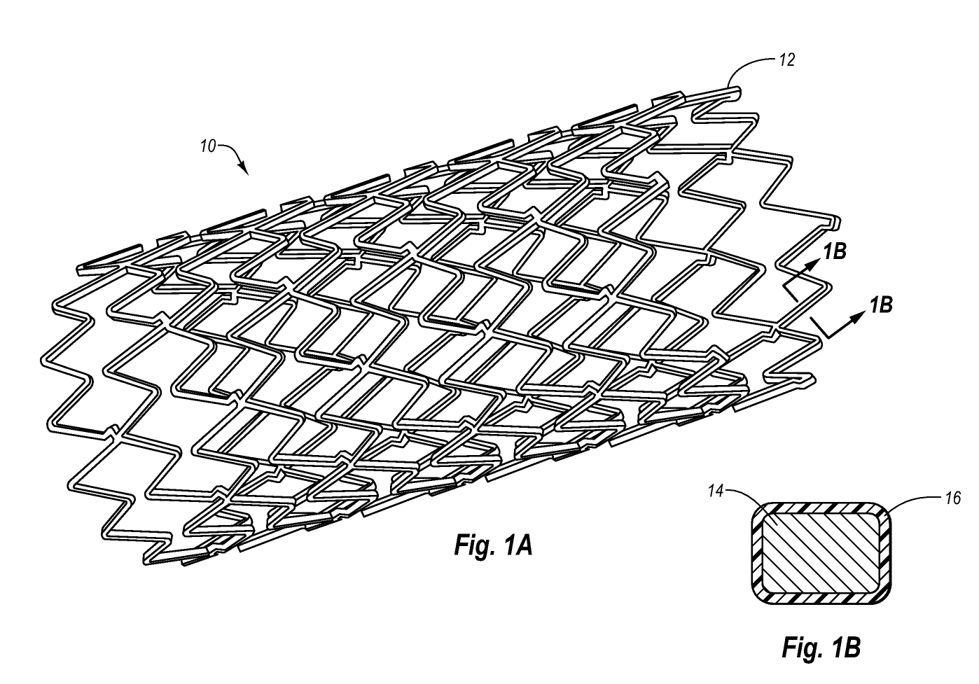

FIG. 1A shows a stent 10 coated with a THV polymer according to one embodiment of the invention. Stent 10 includes a generally tubular body 12 with a lumen. The struts of body 12 (e.g. strut 14) provide a supporting structure for coating the polymers.

FIG. 1B illustrates a cross-section of the stent of FIG. 1A coated with a THV polymer coating 16 according to an embodiment of the invention. The THV polymer coating 16 can be conformal as in FIG. 1B. Alternatively, the coating can be ablumenal, luminal, or any combination thereof Because the THV polymers of the have improved elongation properties compared to existing fluoropolymers, coating 16 can expand as the stent expands during use without cracking.

In one embodiment, a bioactive agent is associated with the coated medical devices. The bioactive agent can be associated with a base coat, top coat, mixed with the THV polymers, and/or be incorporated or otherwise applied to a supporting structure of the medical device.

The bioactive agents can be any moiety capable of contributing to a therapeutic effect, a prophylactic effect, both a therapeutic and prophylactic effect, or other biologically active effect in a mammal. The agent can also have diagnostic properties. The bioactive agents include, but are not limited to, small molecules, nucleotides, oligonucleotides, polynucleotides, amino acids, oligopeptides, polypeptides, and proteins. In one example, the bioactive agent inhibits the activity of vascular smooth muscle cells. In another example, the bioactive agent controls migration or proliferation of smooth muscle cells to inhibit restenosis.

Bioactive agents include, but are not limited to, antiproliferatives, antineoplastics, antimitotics, anti-inflammatories, antiplatelets, anticoagulants, antifibrins, antithrombins, antibiotics, antiallergics, antioxidants, and any prodrugs, metabolites, analogs, homologues, congeners, derivatives, salts and combinations thereof It is to be appreciated that one skilled in the art should recognize that some of the groups, subgroups, and individual bioactive agents may not be used in some embodiments of the invention.

Antiproliferatives include, for example, actinomycin D, actinomycin IV, actinomycin I₁, actinomycin X₁, actinomycin C₁, dactinomycin (COSMEGEN®, Merck & Co., Inc.), imatinib mesylate, and any prodrugs, metabolites, analogs, homologues, congeners, derivatives, salts and combinations thereof Antineoplastics or antimitotics include, for example, paclitaxel (TAXOL®, Bristol-Myers Squibb Co.), docetaxel (TAXOTERE®, Aventis S.A.), midostaurin, methotrexate, azathioprine, vincristine, vinblastine, fluorouracil, doxorubicin hydrochloride (ADRIAMYCIN®, Pfizer, Inc.) and mitomycin (MUTAMYCIN®, Bristol-Myers Squibb Co.), midostaurin, and any prodrugs, metabolites, analogs, homologues, congeners, derivatives, salts and combinations thereof.

Antiplatelets, anticoagulants, antifibrin, and antithrombins include, for example, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors (ANGIOMAX®, Biogen, Inc.), and any prodrugs, metabolites, analogs, homologues, congeners, derivatives, salts and combinations thereof

Cytostatic or antiproliferative agents include, for example, angiopeptin, angiotensin converting enzyme inhibitors including captopril (CAPOTEN® and CAPOZIDE®, Bristol-Myers Squibb Co.), cilazapril or lisinopril (PRINIVIL® and PRINZIDE®, Merck & Co., Inc.); calcium channel blockers including nifedipine; colchicines; fibroblast growth factor (FGF) antagonists, fish oil (omega 3-fatty acid); histamine antagonists; lovastatin (MEVACOR®, Merck & Co., Inc.); monoclonal antibodies including, but not limited to, antibodies specific for Platelet-Derived Growth Factor (PDGF) receptors; nitroprusside; phosphodiesterase inhibitors; prostaglandin inhibitors; suramin; serotonin blockers; steroids; thioprotease inhibitors; PDGF antagonists including, but not limited to, triazolopyrimidine; and nitric oxide; imatinib mesylate; and any prodrugs, metabolites, analogs, homologues, congeners, derivatives, salts and combinations thereof Antiallergic agents include, but are not limited to, pemirolast potassium (ALAMAST®, Santen, Inc.), and any prodrugs, metabolites, analogs, homologues, congeners, derivatives, salts and combinations thereof

Other bioactive agents useful in embodiments of the invention include, but are not limited to, free radical scavengers; nitric oxide donors; rapamycin; methyl rapamycin; 42-Epi-(tetrazoylyl)rapamycin (ABT-578); 40-O-(2-hydroxy)ethyl-rapamycin (everolimus); tacrolimus; pimecrolimus; 40-O-(3-hydroxy)propyl-rapamycin; 40-O-[2-(2-hydroxy)ethoxy]ethyl-rapamycin; tetrazole including rapamycin analogs including those described in U.S. Pat. No. 6,329,386; estradiol; clobetasol; idoxifen; tazarotene; alpha-interferon; host cells including epithelial cells; genetically engineered epithelial cells; dexamethasone; and, any prodrugs, metabolites, analogs, homologues, congeners, derivatives, salts and combinations thereof

Free radical scavengers include, but are not limited to, 2,2′,6,6′-tetramethyl-1-piperinyloxy, free radical (TEMPO); 4-amino-2,2′,6,6′-tetramethyl-1-piperinyloxy, free radical (4-amino-TEMPO); 4-hydroxy-2,2′,6,6′-tetramethyl-piperidene-1-oxy, free radical (TEMPOL), 2,2′,3,4,5,5′-hexamethyl-3-imidazolinium-1-yloxy methyl sulfate, free radical; 16-doxyl-stearic acid, free radical; superoxide dismutase mimic (SODm) and any analogs, homologues, congeners, derivatives, salts and combinations thereof Nitric oxide donors include, but are not limited to, S-nitrosothiols, nitrites, N-oxo-N-nitrosamines, substrates of nitric oxide synthase, diazenium diolates including spermine diazenium diolate and any analogs, homologues, congeners, derivatives, salts and combinations thereof.

The medical devices of the invention can be used in any vascular, non-vascular, or tubular structure in the body. In an embodiment, a coated stent can be used in, but is not limited to use in, neurological, carotid, coronary, aorta, renal, biliary, ureter, iliac, femoral, and popliteal vessels.

### IV. EXAMPLES

The following are specific examples of methods for using THV polymer on a coated implantable device.

### Example 1

Example 1 describes a method for manufacturing a coated stent using THV 220A available from Dyneon of Oakdale, Minn. In a first step, a primer coating is applied to the stent. A primer solution including between about 0.1 mass % and about 15 mass %, (e.g., about 2.0 mass %) of poly(n-butyl methacrylate) (PBMA) and the balance, a solvent mixture of acetone and cyclohexanone (having about 70 mass % of acetone and about 30 mass % of cyclohexanone) is prepared. The solution is applied onto a stent to form a primer layer.

To apply the primer layer, a spray apparatus, (e.g., Sono-Tek MicroMist spray nozzle, manufactured by Sono-Tek Corporation of Milton, N.Y.) is used. The spray apparatus is an ultrasonic atomizer with a gas entrainment stream. A syringe pump is used to supply the coating solution to the nozzle. The composition is atomized by ultrasonic energy and applied to the stent surfaces. A useful nozzle to stent distance is about 20 mm to about 40 mm at an ultrasonic power of about one watt to about two watts. During the process of applying the composition, the stent is optionally rotated about its longitudinal axis, at a speed of 100 to about 600 rpm, for example, about 400 rpm. The stent is also linearly moved along the same axis during the application.

The primer solution is applied to a 15 mm Triplex, N stent (available from Abbott Vascular Corporation) in a series of 20-second passes, to deposit, for example, 20 μg of coating per spray pass. Between the spray passes, the stent is allowed to dry for about 10 seconds to about 30 seconds at ambient temperature. Four spray passes can be applied, followed by baking the primer layer at about 80° C. for about 1 hour. As a result, a primer layer can be formed having a solids content of about 80 μg. For purposes of this invention, “Solids” means the amount of the dry residue deposited on the stent after all volatile organic compounds (e.g., the solvent) have been removed.

In another step, a THV 220A solution is prepared. The solution is prepared by dissolving between about 0.1 mass % and about 15 mass %, (e.g., about 2.0 mass %) of the THV 220A in a solvent. The solvent can be a mixture of about 50 mass % acetone and about 50 mass % dimethylacetamide.

In a manner similar to the application of the primer layer, the copolymer solution is applied to a stent. Twenty spray passes are performed with a coating application of 10 ug per pass, with a drying time between passes of 10 seconds, followed by baking the copolymer layer at about 60° C. for about 1 hour, to form a layer having a solids content between about 30 μg and 750 μg, (e.g., about 225 μg).

### Example 2

Example 2 describes a method for manufacturing a drug eluting stent according to one embodiment of the invention. The medical device is manufactured using the same method as in Example 1, except that instead of the THV 220A solution, a polymer-drug solution is prepared and applied using the following formula.

A drug-including formulation is prepared that includes:
(a) between about 0.1 mass % and about 15 mass %, (e.g., about 2.0 mass %) of THV 220A, available from Dyneon of Oakdale, Minn.;
(b) between about 0.1 mass % and about 2 mass %, for example, about 1.0 mass % of a therapeutic agents. In one embodiment, the therapeutic agent is ABT-578 (available from Abbott Vascular Corp. of Chicago, Ill.); and
(c) the balance, a solvent mixture including about 50 mass % of acetone and about 50 mass % of dimethylacetamide.

The drug-including formulation is applied to the stent in a manner similar to the application of the copolymer solution in Example 1. The process results in the formation of a drug-polymer reservoir layer having a solids content between about 30 μg and 750 μg, (e.g., about 225 μg), and a drug content of between about 10 μg and about 250 μg, (e.g., about 75 μg).

The invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Recitation of ranges of values herein are merely intended to serve as a shorthand method of referring individually to each separate value falling within the range, unless otherwise indicated herein, and each separate value is incorporated into the specification as if it were individually recited herein.

What is claimed is:

1. A medical device comprising a supporting structure having a coating associated therewith, the coating comprising a polymer having the formula, 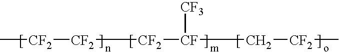
in which,
m is in a range from 0.005 to 0.85;
n is in a range from 0.005 to 0.85;
o is in a range from 0.005 to 0.99; and
m+n+o=1.

2. A medical device as in claim 1, in which the copolymer has a number average molecular weight in a range from about 20K to about 800K.

3. A medical device as in claim 1, in which the copolymer has a number average molecular weight in a range from about 100K to about 600K.

4. A medical device as in claim 1, in which the polymer has an elongation at break in a range from about 50% to about 800%.

5. A medical device as in claim 1, in which the polymer has an elongation at break in a range from about 100% to about 700%.

6. A medical device as in claim 1, in which the polymer has an elongation at break in a range from about 300% to about 800%.

7. A medical device as in claim 1, in which n is in a range from about 0.005 to about 0.75.

8. A medical device as in claim 1, in which n is in a range from about 0.005 to about 0.5.

9. A medical device as in claim 1, in which the supporting structure is selected from a group consisting of coronary stents, peripheral stents, catheters, arterio-venous grafts, by-pass grafts, pacemaker and defibrillator leads, anastomotic clips, arterial closure devices, patent foramen ovale closure devices, and drug delivery balloons.

10. A medical device as in claim 1, in which the supporting structure comprises a stent that is self expandable.

11. A medical device as in claim 1, in which the supporting structure comprises a stent that is balloon expandable.

12. A medical device as in claim 1, in which at least one therapeutic agent is associated with the copolymer.

13. A medical device as in claim 12, in which the at least one bioactive agent is associated with a top coat, a bottom coat, a portion of the structure of the medical device, or a combination thereof

14. A medical device as in claim 12, in which the at least one bioactive agent is an anti-proliferative, anti-inflammatory, antineoplastic, antiplatelet, anti-coagulant, anti-fibrin, antithrombonic, antimitotic, antibiotic, antiallergic or antioxidant drug.

15. A medical device as in claim 12, in which the anti-inflammatory drug is steroidal or non-steroidal.

16. A medical device as in claim 1, in which the coating is applied using a powder coating technique.

17. A method for using a THV terpolymer on a medical device, comprising:
dissolving a terpolymer of poly(tetrafluoroethylene-co-hexafluoropropylene-co-vinylidene fluoride) in an organic solvent to form a coating mixture;
coating an implantable medical device with the coating mixture; and
removing the organic solvent from the coating mixture to produce a substantially solvent-free coating.

18. A method as in claim 17, in which the copolymer solution is applied using spraying, dip coating, roll coating, spin coating, direct application by brush or needle, or a combination thereof

19. A method as in claim 17, in which the organic solvent comprises a ketone, ester, ether, amide, or combination thereof

20. A method as in claim 17, in which the solvent is selected from the group consisting of dimethylacetamide (DMAC), dimethylformamide (DMF), tetrahydrofuran (THF), dimethylsulfoxide (DMSO), cyclohexanone, xylene, toluene, acetone, i-propanol, methyl ethyl ketone, propylene glycol monomethyl ether, methyl t-butyl ketone, methyl isobutyl ketone, ethyl acetate, n-butyl acetate, n-butanol, ethanol, methanol, chloroform, trichloroethylene, 1,1,1-trichloreoethane, methylene chloride, dioxane, and mixtures thereof.

21. A method as in claim 17, in which the solvent is a mixture selected from the group consisting of DMAC and methanol (50:50 w/w); i-propanol and DMAC (80:20, 50:50, or 20:80 w/w); acetone and cyclohexanone (80:20, 50:50, or 20:80 w/w); acetone and xylene (50:50 w/w); acetone, xylene and FLUX REMOVER AMS® (93.7% 3,3-dichloro-1,1,1,2,2-pentafluoropropane and 1,3-dichloro-1,1,2,2,3-pentafluoropropane, and the balance is methanol with trace amounts of nitromethane; Tech Spray, Inc.) (10:40:50 w/w); and 1,1,2-trichloroethane and chloroform (80:20 w/w).

22. A method as in claim 17, in which the medical device is selected from the group consisting of coronary stents, peripheral stents, self expanding stents, catheters, arterio-venous grafts, by-pass grafts, pacemaker and defibrillator leads, anastomotic clips, arterial closure devices, patent foramen ovale closure devices, and drug delivery balloons.

23. A method as in claim 17, in which the supporting structure comprises a stent that is self expandable.

24. A method as in claim 17, in which the supporting structure comprises a stent that is balloon expandable.

25. A method as in claim 17, in which the copolymer has a number average molecular weight in a range from about 20K to about 800K.

26. A method as in claim 17, in which the copolymer has a number average molecular weight in a range from about 100K to about 600K.

27. A method as in claim 17, in which the polymer has an elongation at break in a range from about 50% to about 800%.

28. A method as in claim 17, in which the polymer has an elongation at break in a range from about 100% to about 700%.

29. A method as in claim 17, in which the polymer has an elongation at break in a range from about 300% to about 800%.

30. A method as in claim 17, in which n is in a range from about 0.005 to about 0.75.

31. A method as in claim 17, in which n is in a range from about 0.005 to about 0.5.

32. A method as in claim 17, in which the medical device is coated using spraying, dip coating, roll coating, spin coating, inkjet printing, direct application by brush or needle, or a combination thereof.

33. A method as in claim 17, in which at least one bioactive agent is associated with the medical device.

34. A method as in claim 33, in which the at least one bioactive agent is associated with a top coat, bottom coat, or the supporting structure.

35. A method as in claim 34, in which the at least one bioactive agent is an anti-proliferative, anti-inflammatory, antineoplastic, antiplatelet, anti-coagulant, anti-fibrin, antithrombonic, antimitotic, antibiotic, antiallergic or antioxidant drug.

36. A medical device manufactured according to any of claims 17 to 35.
